(19) United States
(12) Patent Application Publication
(10) Pub. No.: US 2020/0157493 A1
(43) Pub. Date: May 21, 2020

(54) TEXTILE GROWTH MATRIX FOR CELLS

(71) Applicant: THE SECANT GROUP, LLC, Telford, PA (US)

(72) Inventors: Brian GINN, Chalfont, PA (US); Peter D. GABRIELE, Frisco, PA (US); Jeremy J. HARRIS, Doylestown, PA (US); Amanda K. WEBER, Macungie, PA (US)

(21) Appl. No.: 16/691,040

(22) Filed: Nov. 21, 2019

Related U.S. Application Data
(60) Provisional application No. 62/770,509, filed on Nov. 21, 2018.

(51) Int. Cl. C12N 5/00; D04B 21/08; D04B 21/20

(52) U.S. Cl. CPC C12N 5/0068; D04B 21/08; D04B 21/202; C12N 2533/50; C12N 2533/40; C12N 2533/90; C12N 2539/00; C12N 2535/00

(57) ABSTRACT

A engineered textile construction includes a first textile having a first average pore size forming a textile cell growth matrix in which the first textile is a woven or a knit construction, the textile cell growth matrix is configured to have a surface area sufficient to promote cell expansion and the first average pore size is preselected to prevent filling of the pores during cell expansion.

### RELATED APPLICATIONS

This application claims the benefit of, and priority to, U.S. application Ser. No. 62/770,509 filed Nov. 21, 2018, which is hereby incorporated by reference in its entirety.

### FIELD

The present invention is directed to textile-based cell growth matrix structures for use in cell-based bioprocessing applications. More particularly, the present application is directed to woven and knit cell growth matrices having a uniform pore size.

### BACKGROUND

Conventional structures used in bioreactors as cell supports are based on nonwoven sheets. Nonwoven sheets offer a one-size-fits-all approach to providing supports for cells of different types which suffer from a number of limitations resulting from this approach.

The disordered structure of non-woven supports can result in variations in cell response and growth throughout due to the inhomogeneity of the pore structure, leading to non-optimal cell growth. Additionally, typical non-woven textile-based structures are prone to fragmentation/shedding during cell expansion, their treatment, or collection of cells. These fragments need to be removed before cells can be introduced back into a patient for cell-based therapy applications or isolated to improve purity of harvested virus, antibodies, or other biologics.

For example, a conventional disk structure is a non-woven polyethylene terephthalate (PET) mesh backed by large polypropylene (PP) filaments. The disks are prone to fragmentation due to their non-woven structure. Folded, non-disk, polyester non-wovens are another typical configuration, but have the same limitations as non-woven disks. The non-woven structural porosity is also typically filled as cells proliferate on the substrate. The density of the structure described by the current art already severely limits nutrient flow through it as the bulk of flow occurs around as opposed to penetrating and flowing though the structure. The proliferating cells only continue to decrease porosity, which further limits cellular proliferation.

Additionally, access to media can become inconsistent with conventional non-woven textile supports due to irregular pore structures and/or their planar nature leading to a filling of smaller pores as cells proliferate. The closing of pores during cell expansion alters the cell media flow dynamics and limits nutrient access to the proliferating cells in the interior of the support. This limitation over the course of cell processing results in non-optimal access to nutrients for cell growth/expansion or inconsistent access to soluble factors that control cell differentiation, phenotype, and viability.

### SUMMARY

In an embodiment, an engineered textile construction includes a first textile having a first average pore size forming a textile cell growth matrix. The first textile is a woven or a knit construction, the textile cell growth matrix is configured to have a surface area sufficient to promote cell expansion and the first average pore size is preselected to prevent filling of the pores during cell expansion.

In another embodiment, a cell culture system comprises a container that includes the engineered textile constructions described herein in combination with at least one culturable cell type and a cell culture medium.

Features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 illustrates a textile construction, according to an embodiment.

FIG. 2 illustrates a textile construction, according to an embodiment.

FIG. 3 illustrates a textile construction, according to an embodiment.

FIG. 4 illustrates a textile construction, according to an embodiment.

FIG. 5 illustrates a textile construction, according to an embodiment.

FIG. 6 illustrates a textile construction, according to an embodiment.

FIG. 7 illustrates a textile construction, according to an embodiment.

FIG. 8 illustrates a textile construction, according to an embodiment.

FIG. 9 illustrates a textile construction, according to an embodiment.

FIG. 10 illustrates a textile construction, according to an embodiment.

FIG. 11 illustrates a system for cell culture, according to an embodiment.

FIG. 12 illustrates a textile construction in a single use bioreactor system, according to an embodiment.

FIG. 13 illustrates a textile construction in a single use bioreactor system, according to an embodiment.

FIG. 14 illustrates retrieval of cells from a single use bioreactor according to an embodiment.

FIG. 15 illustrates a stacked textile construction, according to an embodiment.

FIG. 16 illustrates culture of human cardiac fibroblasts on textile cell growth matrices, according to an embodiment.

FIG. 17 is a graph of glucose and lactate concentrations over time during cell proliferation.

Wherever possible, the same reference numbers will be used throughout the drawings to represent the same parts.

### DETAILED DESCRIPTION

To address these shortcomings in the art, provided are woven, knit, or braided engineered textiles that provide a growth matrix for cells having with tunable porosity for bioprocessing use as a high surface area substrate to increase yield during cellular expansion, microbial culture, or viral production. The engineered textiles incorporate structural or coating properties that are tuned for specific cell types leading to improved cellular outcomes compared to current commercial products. This can include modification of physiochemical properties such as matrix or coating stiffness and surface chemistry of the textile growth matrix or cell type specific coatings to enhance attachment, proliferation, and function.

Contour guidance is the natural propensity for growing tissue cells to follow the contour features of a surface as the tissue expands. During bioreactor cell growth, the tissue expands to colonize the cellular growth matrix, as part of the growth process. The colonization into a textile structure, such as the cellular growth matrix, which may act as scaffold or template, is a form of bonding mechanism of textile to tissue. The choice of matrix structure may be customized to optimize cell growth and colonization based on cell type.

The growth matrix incorporates woven, knit, and braided textile structures to generate a more ordered structure that can be engineered to better produce outcomes when culturing large numbers of cells in a packed bed bioreactor. Specific applications arise from the response by cells to particular structures that can be produced with these textile technologies that cannot be generated with the disordered structure from conventional non-woven fiber technologies.

The cellular growth matrices may be formed from various woven, knit, or braided constructions, including a double needle bar knit, a plain weave, twill weave, rib weave (e.g., warp rib or weft rib), satin weave, mock leno weave, and/or herringbone weave. In some embodiments, the cellular growth matrices are formed from a plain weave, a leno weave, or knit constructions. In one embodiment, the cellular growth matrices are formed from a leno weave construction. In one embodiment, the cellular growth matrices are formed from a double needle bar (DNB) knit construction. In some embodiments, cellular growth matrices are formed from a plurality of the above constructions.

In an embodiment, the woven, braided, or knit structure may include filaments, fibers, or yarns having differing fiber cross-sections. In some embodiments, the cross sections may include circular, elliptical, multi-lobal (e.g., trilobal, tetralobal), triangular, lima bean, lobular, flat, and/or dog-bone cross-sections. In some embodiments, the cross sections may further be serrated. In some embodiments, the fibers and/or yarns may be continuous filament and/or multifilament fibers, or yarns. In some embodiments, the textile growth matrices may be formed using monofilament yarns. In some embodiments, the textile growth matrices may be formed using multifilament yarns. In some embodiments, the textile growth matrices may be formed using multiple configurations of plied yarns. In some embodiments, the cellular growth matrices may include fibers having fractal fiber designs (as described in U.S. Pub. 2011/0076771 incorporated by reference herein), sheath-core, or islands-in-the-sea type cross-sections.

The cellular growth matrices may be a single or multi-layered textile. Woven, knit, and braided structures better organize the individual yarns or filaments relative to a non-woven growth matrix. Such engineered textile structures have fewer loose ends.

Multilayered woven textile structures additionally provide “pockets” within the structure in which cells may be partially protected from high solution shear effects within a stirred or perfusion bioreactor. Multilayered or stacked structures also provide benefits over the current art as they support cellular growth in three-dimensions (3-D) opposed to the flat, planar two-dimensional (2-D) structures used conventionally.

The porosity of the textile structure may be controlled during the material selection and construction process. Tunable porosity improves nutrient access to cells and therefore proliferation rate and cell health in bioreactor culture. The porosity of the growth matrix may be selected based on the type of cells being grown in the bioreactor. In some embodiments, the textile structure may additionally be pre-seeded with a culturable cell type prior to exposure to the nutrient.

The porous woven textile structures are modified through alterations in pick and end spacings to tune cell access to nutrients. Localized alignment, as experienced by the cells, in woven textiles are altered through ribbing, twilling, and other weaving techniques. Porosity can range from almost no porosity to highly porous structures. In general, high porosity growth matrices are best used with self-aggregating cells that form three-dimensional (3D) clusters. Self-aggregating cell colonies are generally formed on a low adhesion substrate or through buildup of cells beyond a monolayer. High porosity growth matrices may provide space for aggregates to form once a single layer of cells colonizes the underlying textile growth matrix. In an alternate embodiment, 3-D printing can generate 3D porous structures.

In some embodiments, self-aggregating cells include neural stem cells, mesenchymal stem cells (MSCs), hepatocytes, pancreatic islet cells, induced pluripotent stem cells (iPSCs), human umbilical vein endothelial cells (HUVEC), adipose derived stem cells (ASCs), human embryonic kidney (HEK 293), and embryoid bodies. In yet other embodiments, self-aggregating cells include tumor cells, carcinoma cells, and sarcoma cells including human breast adenocarcinoma cell line (MCF-7), liver hepatocellular carcinoma (HepG2), Y79 retinoblastoma cells. In some embodiments, non-human self-aggregating cells include COS-7 simian cells, Sf9 insect cells, Chinese hamster ovary cells (CHO), baby hamster kidney cells (BHK), and mouse 3T3 fibroblast cell lines. In some embodiments, self-aggregating cells include hybridomas.

In some embodiments, the porosity of a layer and/or the overall textile growth matrix may be at least 5 percent, at least 10 percent, at least 15 percent, at least 20 percent, at least 25 percent, at least 30 percent, at least 35 percent, at least 40 percent, at least 45 percent, less than 75 percent, less than 70 percent, less than 65 percent, less than 60 percent, less than 55 percent, less than 50 percent, and combinations of ranges and sub-ranges thereof.

The cellular growth matrix may be formed having a range of surface areas sufficient to support cellular expansion. In general, more porous textile structures have increased surface area. In some embodiments, the surface area of a layer and/or the overall textile growth matrix may be at least 0.01 meter squared per gram (m²/g), at least 0.1 m²/g, at least 0.5 m²/g, at least 1.0 m²/g, at least 1.5 m²/g, less than 10.0 m²/g, less than 8.0 m²/g, less than 6.0 m²/g, less than 5.0 m²/g, less than 4.0 m²/g, less than 3.0 m²/g, less than 2.5 m²/g, less than 2.0 m²/g, and ranges and subranges thereof.

In some embodiments, the average pore size may be between 0 micrometers to 2 millimeters, 2 micrometers to 1 millimeter, 5 micrometers to 800 micrometers, 10 micrometers to 600 micrometers, 15 micrometers to 500 micrometers, 20 micrometers to 400 micrometers, 30 micrometers to 350 micrometers, 40 micrometers to 300 micrometers, 50 micrometers to 250 micrometers, 60 micrometers to 200 micrometers, 70 micrometers to 170 micrometers, 80 micrometers to 150 micrometers, 90 micrometers to 130 micrometers, 100 micrometers to 120 micrometers, and ranges and subranges thereof. The average pore size of the layers of a multi-layer growth matrix may be the same or different.

The pore size may be selected based on the cell colonies being formed. Large pore sizes allow nutrients to easily flow throughout the growth matrix structure and provide large open areas for cells to proliferate. Self-aggregating cell culture may produce high yields in a porous environment. Lower porosity textile growth matrices may promote the culture of non-self-aggregating cell types, such as epithelial cells. In some embodiments, an average pore size of about 30 micrometers to about 350 micrometers may allow for the efficient culture of various cell types. Pore size may range into the mm range, but such large pore sizes generally result in reduced surface area available for cell expansion.

Knitting porosity can be controlled through selection of yarn denier and loop size. The extensive porosity of knit structures allows cells to form aggregates as expansion proceeds and cell numbers build up to beyond levels of a confluent monolayer. In some embodiments, knit growth matrices are used in the formation of cell spheroids, such as result from the growth of neural stem cells, mesenchymal stem cells, tumor cells, such as cancer cells, mesenchymal stem cells, pancreatic islet cells, and induced pluripotent stem cells, and other self-aggregating cell types.

In some embodiments, the yarn denier of the braided, knit, or woven growth matrix is at least 5 denier, at least 7 denier, at least 10 denier, at least 12 denier, at least 15 denier, at least 20 denier, at least 30 denier, at least 50 denier, at least 80 denier, less than 1000 denier, less than 750 denier, less than 500 denier, less than 250 denier, less than 200 denier, less than 100 denier, and ranges and subranges thereof.

In some embodiments, the average knit loop size is at least 150 micrometers, at least 200 micrometers, at least 250 micrometers, at least 300 micrometers, at least 350 micrometers, at least 400 micrometers, less than 550 micrometers, less than 500 micrometers, less than 450 micrometers, and ranges and subranges thereof.

In some embodiments, the textile growth matrix possesses shape consistency throughout the structure. By shape consistency it is meant a substantially uniform pore size throughout the textile structure. Shape consistency improves consistency and control of the textile structure to which the cells are attached, resulting in improved uniformity of the rate of cell growth.

The braided, woven, or knit growth matrix may exhibit a thickness that is dependent upon the desired cell type growth in the bioreactor. In some embodiments, the growth matrix thickness is substantially uniform across the face of the textile. In some embodiments, the thickness of textile may be at least 35 micrometers, at least 40 micrometers, at least 42 micrometers, at least 45 micrometers, at least 50 micrometers, at least 70 micrometers, at least 100 micrometers, at least 120 micrometers, at least 150 micrometers, at least 200 micrometers, less than 1000 micrometers, less than 900 micrometers, less than 800 micrometers, less than 700 micrometers, less than 650 micrometers, less than 600 micrometer, less than 550 micrometers, less than 500 micrometers, less than 450 micrometers, less than 400 micrometers, less than 350 micrometers, less than 300 micrometers, less than 250 micrometers, and ranges and subranges thereof.

In some embodiments, the textile growth matrix may include multiple textile layers. In some embodiments, the thickness of the layers of the multi-layer growth matrix may be at least 35 micrometers, at least 40 micrometers, at least 42 micrometers, at least 45 micrometers, at least 50 micrometers, at least 70 micrometers, at least 100 micrometers, at least 120 micrometers, at least 150 micrometers, less than 350 micrometers, less than 300 micrometers, less than 250 micrometers, and combinations thereof.

The braided, woven, or knit growth matrix may be formed from any resorbable material, non-resorbable material, or combination of materials suitable for textile forming. Suitable non-resorbable materials include, but are not limited to, poly(ethylene terephthalate) (PET), polypropylene (PP), poly(vinylidene fluoride) (PVDF), silicone, polyurethane, polycarbonate, polyether ketone, collagen, fibronectin, hyaluronic acid, and combinations thereof. Suitable resorbable materials include, but are not limited to, polycaprolactone (PCL), polylactic acid (PLA), polyglycolic acid (PGA), poly(lactic-co-glycolic acid) (PLGA), poly(glycerol sebacate) (PGS), lysine-poly(glycerol sebacate) (KPGS), acrylated poly(glycerol sebacate) (PGSA), poly(trimethylene carbonate) (PTMC), poly(dioxanone) (PDO), collagen, fibrin, alginate, silk, and combinations thereof. In some embodiments, the growth matrix may include polyethylene terephthalate (PET). In one embodiment, the growth matrix may be formed from polyethylene terephthalate (PET). In one embodiment, the growth matrix includes PET fiber having a tenacity greater than 7 grams per denier (7 g/den). In one embodiment, the growth matrix includes a PET fiber having a round profile.

In some embodiments a coating may be provided to the fibers or yarns of the growth matrix. In some embodiments, the coating may be applied to the fibers or yarns prior to the formation of the textile. In some embodiments, the coating may be applied after formation of the textile structure. In some embodiments, the coating may be formed from resorbable materials. The resorbable materials may enhance endogenous regeneration of tissue. Suitable resorbable materials include, but are not limited to, polycaprolactone (PCL), polylactic acid (PLA), polyglycolic acid (PGA), poly(glycerol sebacate) (PGS), lysine-poly(glycerol sebacate) (KPGS), poly(glycerol sebacate urethane) (PGSU), amino-acid incorporated PGS, acrylated poly(glycerol sebacate) (PGSA) and combinations thereof. In some embodiments, the coatings may be applied by spray or dip coating, or lamination. The coating may improve cellular attachment to the growth matrix and reduce risk of residual textile fragment formation. The resorbable materials additionally provides a biodegradable substrate that can be injected into the patient along with the cells should some of the coating delaminate during the trypsinization process. Other variants of PGS may also be used including coating with alternative amino acid functionalized PGS compositions, PGS compositions utilizing alternative crosslinking motifs such as urethane linkages.

In an embodiment, the cell growth matrix may be used for in vivo cell-based therapy applications where the cultured cells are intended to provide a regenerative function for damaged tissue or organ system. Due to the bioresorbable and biocompatible nature of the cell growth matrices of some embodiments as described herein, the growth matrix can be implanted following cell expansion at an anatomical site specific to the cellular payload. For example, a textile growth matrix composed of insulin producing cells is implanted in or adjacent to the pancreas as to facilitate the regeneration of the insulin producing capabilities of a compromised pancreas. For example, a textile growth matrix containing cardiac myocytes or progenitor cells can be implanted at or proximate to the site of a cardiac infarction to regenerate native cardiac tissue.

In an embodiment, textures and surface features may be added to the textile by post-production processing. After the formation of the textile, the textile may be subjected to additional processing, such as laser ablation, laser etching, chemical etching, corona treatment, or plasma treatment. Post-production processing may add various features including micro-via through hole structures, surface texture modifications (e.g., surface roughness), and/or patterning. The textile growth matrix may optionally be additionally laser cut or heated along the edges to secure (melt) together fiber ends. The textile growth matrix may also be produced using fibers with heat shrinkage properties to produce crinkled or other 3D textile structures.

In some embodiments, the post-production processing may enhance cellular attachment and protein adsorption. In some embodiments, the textile growth matrix is coated with cell binding proteins such as collagen, fibronectin, laminin, -RGD (amino acid sequence: arginine-glycine-aspartate) containing peptides, -IKVAV (amino acid sequence: isoleucine-lysine-valine-alanine-valine) containing peptides, and -YIGSR (amino acid sequence: tyrosine-isoleucine-glycine-serine-arginine) containing peptides. In some embodiments, the textile growth matrix is coated with positively charged materials such as poly-lysine. In some embodiments, soluble factor sequestering molecules, such as heparin, are conjugated to the textile growth matrix surface. In some embodiments, glycosaminoglycans (GAGs) or polysaccharides, such as hyaluronic acid, are incorporated onto the surface of the textile growth matrix.

By incorporating topography (e.g., patterning and/or texturing) to the surface of the implanted textile, the developed surface provides an integrated guidance structure for the colonizing cells to follow. This creates a more secure textile to tissue bonding relationship by the high degree of tissue in-growth into the textile.

The use of braided, woven, and/or knit cell growth matrices eliminates the need for additional processing steps with the extracted cells. There is no longer fragmentary material that needs to be separated from the extracted cells as is needed with the use of conventional growth matrix materials. Conventional products such as FibraCelTM disks or BioNOCII carriers, are composed of two different fiber materials at significantly different sizes. In some embodiments, the textile growth matrix are composed of a single material type which provides an advantageous and consistent growth environment for cells.

### EXAMPLES

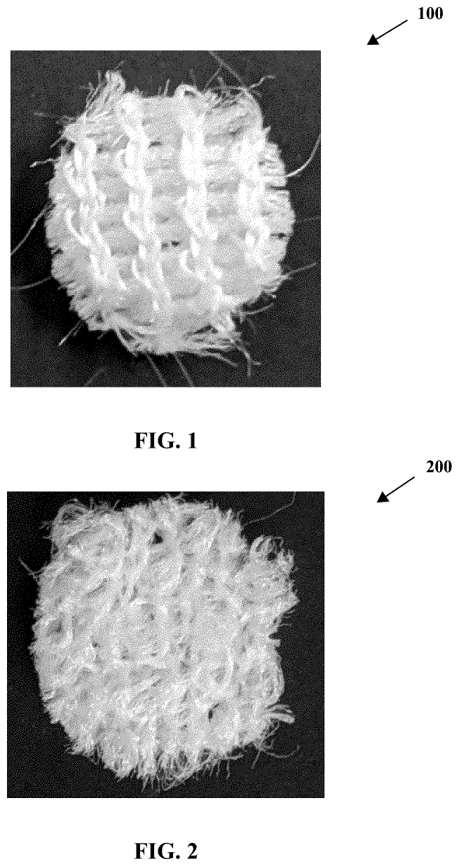

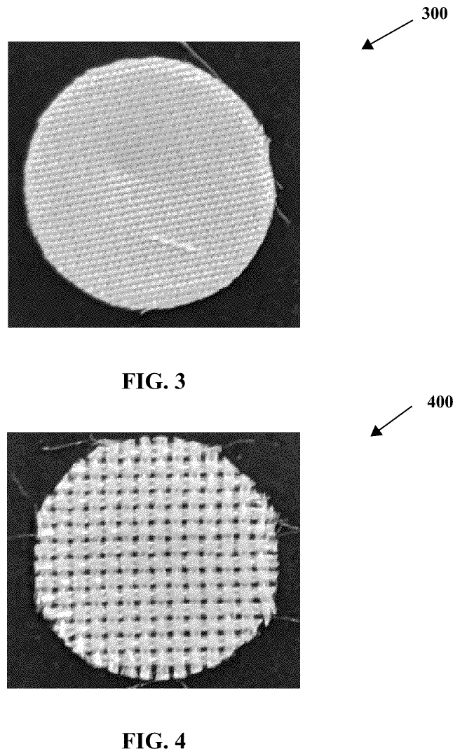

Various exemplified example embodiments are presented in the FIGS. In the examples of FIGS. 1-4 the illustrated growth matrices are uncoated. The samples have been cut into about 6 millimeter diameter disks. FIG. 1 illustrates a double needle bar (DNB) knit construction 100. FIG. 2 illustrates a multi-layer woven construction 200. FIG. 3 illustrates a porous mock-leno weave construction 300. FIG. 4 illustrates a texturized double needle bar (DNB) knit construction 400. The illustrated examples employ PET as the fiber material. Any of the fibrous materials described above may also be used alone or in combination in the formation of the growth matrices.

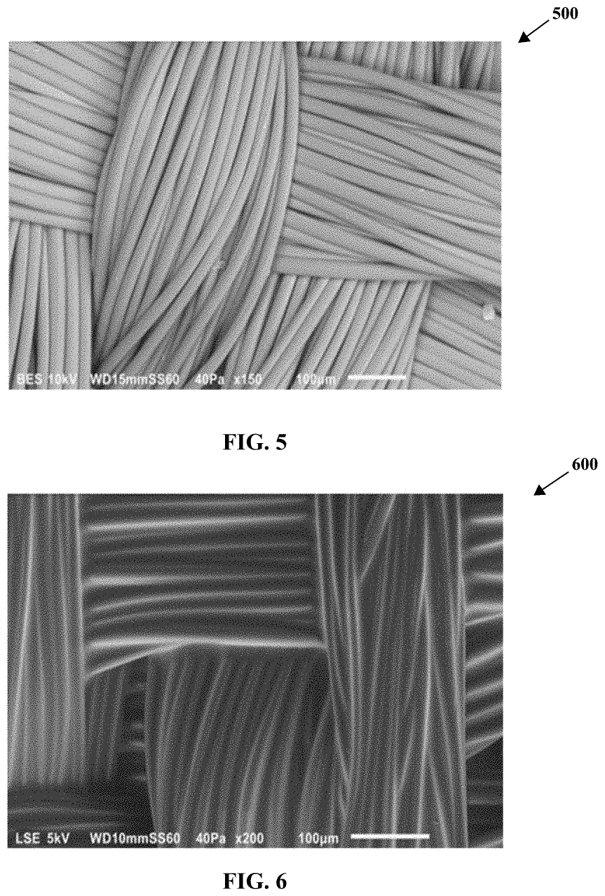

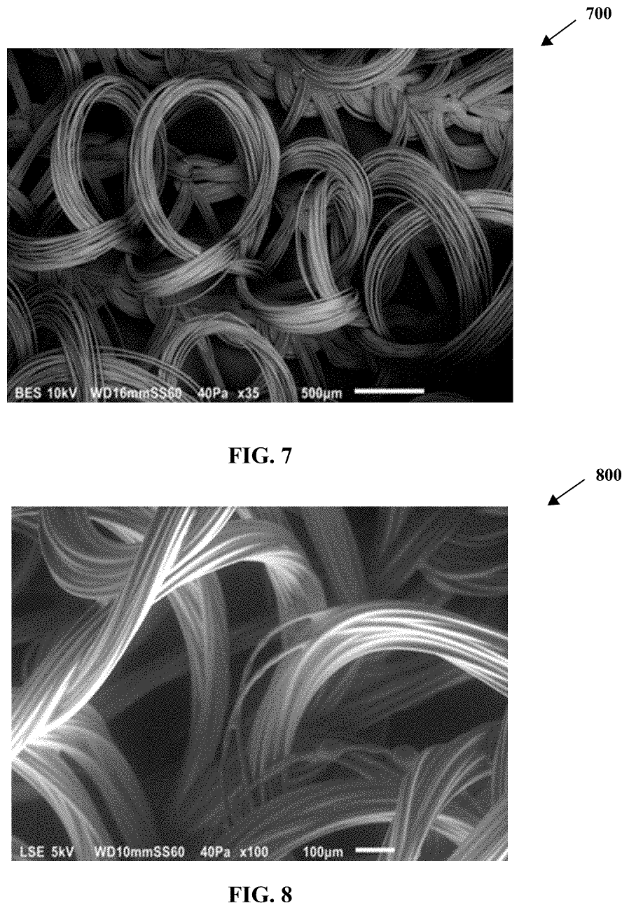

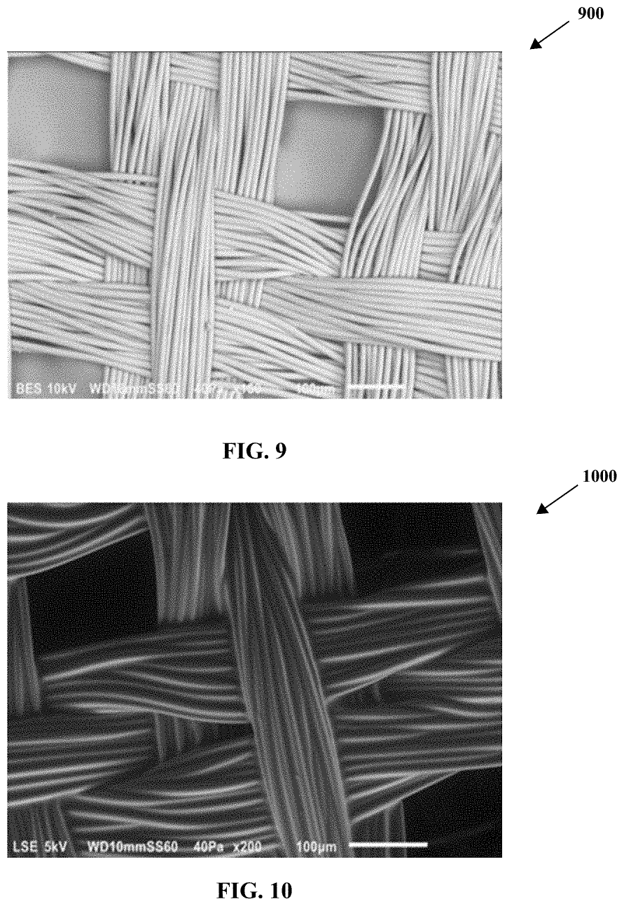

The examples of FIGS. 5-10 provide scanning electron microscope images of various embodiments. FIG. 5 is an uncoated orthogonal weave construction 500 having low porosity. FIG. 6 is a PGS coated orthogonal weave construction 600 having low porosity. FIG. 7 is an uncoated double needle bar (DNB) knit construction 700 having high porosity. FIG. 8 is a PGS coated double needle bar (DNB) knit construction 800 having high porosity. FIG. 9 is an uncoated mock leno weave construction 900 having moderate porosity. FIG. 10 is a PGS coated mock leno weave construction 1000 having moderate porosity.

The disordered structure of non-wovens can result in small variations in cell response and growth throughout the growth matrix due to the inhomogeneity of the pore structure, leading to non-optimal growth conditions. By tuning the pore structure, these variations can be eliminated. Control over pore architecture that results from using engineered woven, knit, and braided textiles allows improved modeling of cell culture media flow dynamics that can be used to tune textile growth matrix properties. Other technologies such as 3D printing can generate 3D porous structures, but these are limited towards larger pore sizes at low surface area to volume ratios.

Additionally, conventional non-woven structures are limited in that they are mechanically weak unless supported by a secondary fiber support structure. This results in additional processing to generate the growth matrix and should the support fiber delaminate, significant deterioration of the growth matrix could occur, requiring removal from the bioreactor. The exemplified textile growth matrices do not require a secondary growth matrixing be fused onto the main culture structure, reducing components that may provide avenues of growth matrix failure.

Cell media flow through the engineered textiles is improved via use of controlled pore architecture. Examples of suitable constructions include the mock leno structure, shown in FIGS. 9 and 10, that provides large, controlled sized pores, that are of sufficient size to prevent cells from covering the pores as they proliferate.

An exemplary embodiment includes the use of a woven textile structure with pore size sufficiently large to prevent filling as cells proliferate. Structures in which the pores fill during cell proliferation may result in reduced transport of nutrients contained within the culture media, resulting in reduced cell expansion efficiency. The embodiment is mechanically robust enough to not require a secondary fiber to provide additional structural support to the main growth matrix structure. This embodiment is additionally coated with lysine-PGS (KPGS) to improve cellular attachment and further reduce the risk of substrate shedding during processing and use. An example of a mock leno bioreactor growth matrix having a large pore size is shown in FIG. 4.

In an exemplary embodiment, mock leno bioreactor growth matrices are 6 millimeter diameter disks with a thickness of about 500 micrometers composed of multifilament polyethylene terephthalate (PET) yarns that are spray coated with lysine-poly(glycerol sebacate) (KPGS) after KPGS is solubilized in THF. The coating is then thermoset at 120° C. for 24 hours in a vacuum oven at 10 torr. During use, KPGS degrades over the course of cell culture allowing cell growth upon the underlying textile structure while initially filling the spacing between individual yarn fibers. The coating of KPGS is sufficiently thin that the underlying textile structure remains visible and thus maintains the additional surface area available for cellular attachment that stems from the yarn thread architecture. Disk shapes are prepared via laser cutting or mechanical cutting using a die. An example of a mock leno bioreactor growth matrix having coated fibers is shown in FIG. 10. Textile growth matrices in accordance with embodiments described herein may also be provided in other geometries such as squares, ovals, tubes, and other shapes. Textile growth matrices in accordance with embodiments described herein may also be made into large sheets that may be used in a bioreactor device as a single piece or multiple large pieces.

The textile structure may be used as a non-implantable cell culture growth matrix in a packed bed bioreactor. This includes embodiments in the form of disks that are freely packed together or as larger sheets that are placed into single use bioreactor reactor bags. These disks have a highly engineered structure to provide a more uniform environment for cell culture with reduced incidence of fragmentation and flexibility to be modified through addition of PGS coatings. The yarns or fibers that make up a textile cell growth matrix can be texturized to yield even more porosity and add additional three-dimensionality to the structure to better utilize the potential volume in a bioreactor for cell culture.

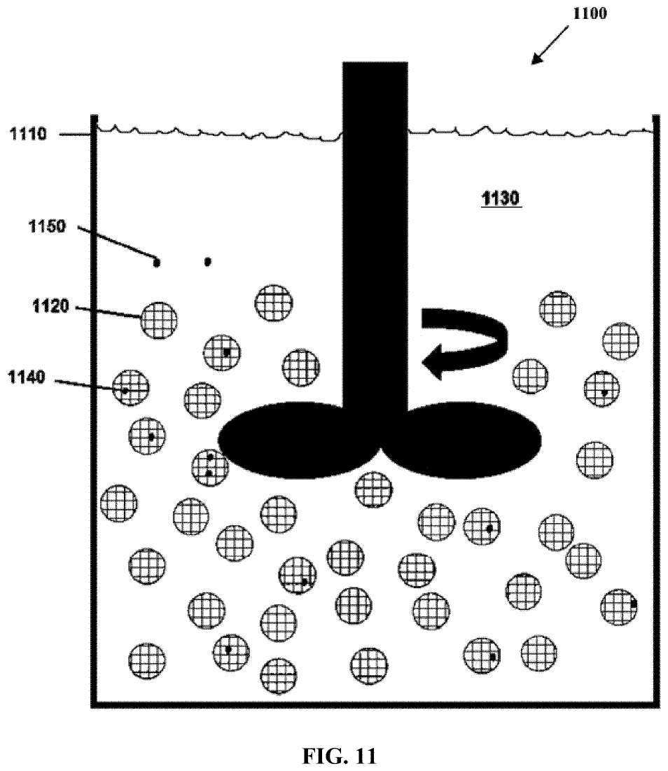

FIG. 11 illustrates a cell culture system 1100 having a bioreactor 1110 containing textile cell growth matrices 1120 and a cell growth medium 1130. Cell colonies 1140 may be formed on the textile cell growth matrix 1120 by the proliferation of culturable cells 1150.

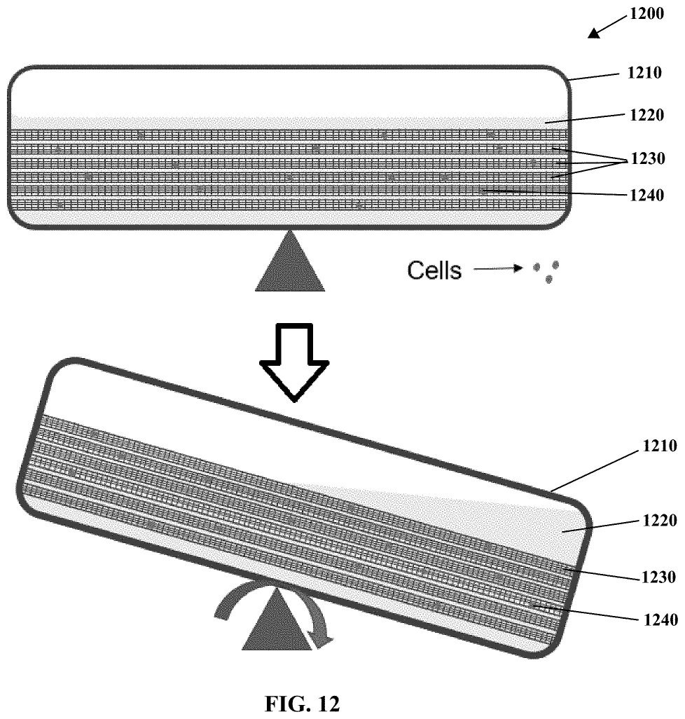

In some embodiments, layers of textile cell growth matrices are placed within single use bioreactors to increase the surface area available for cell culture or to isolate distinct cell populations between textile layers. An example embodiment is shown in FIG. 12. In the example of FIG. 12, a bioreactor system 1200 includes a bioreactor 1210 containing a nutrient rich medium 1220 further containing a plurality of distinct textile cell growth matrices 1230 on which cell colonies 1240 may proliferate. In the example of FIG. 12 the bioreactor 1210 may undergo a rocking motion to assist in the fluid flow of the nutrient rich medium 1220 through the textile cell growth matrix 1230.

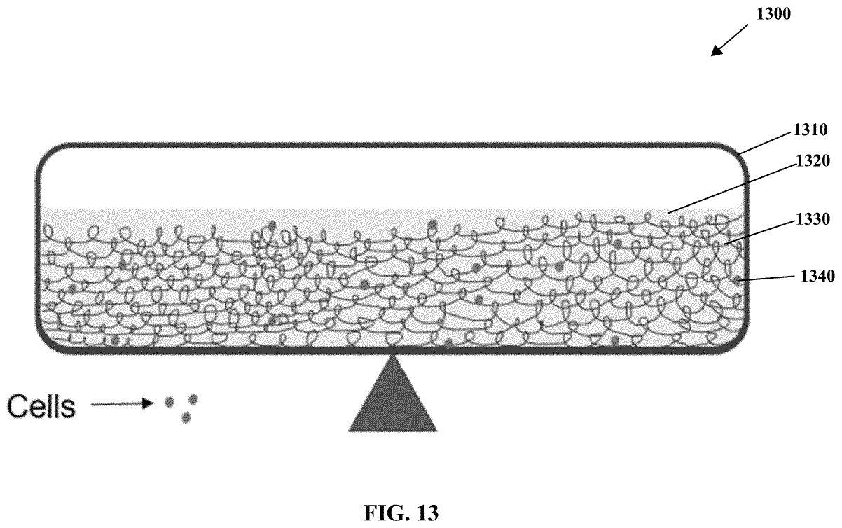

In another embodiment, the textile growth matrix may be a continuous phase within the space available in a single use bioreactor such as the example embodiment of a 3D knit structure as shown in FIG. 13. In the example of FIG. 13, a bioreactor system 1300 includes a bioreactor 1310 containing a nutrient rich medium 1320 further containing a continuous textile cell growth matrix 1330 on which cell colonies 1340 may proliferate. In the example of FIG. 13 the bioreactor 1310 may undergo a rocking motion to assist in the fluid flow of the nutrient rich medium 1320 through the continuous textile cell growth matrix 1330.

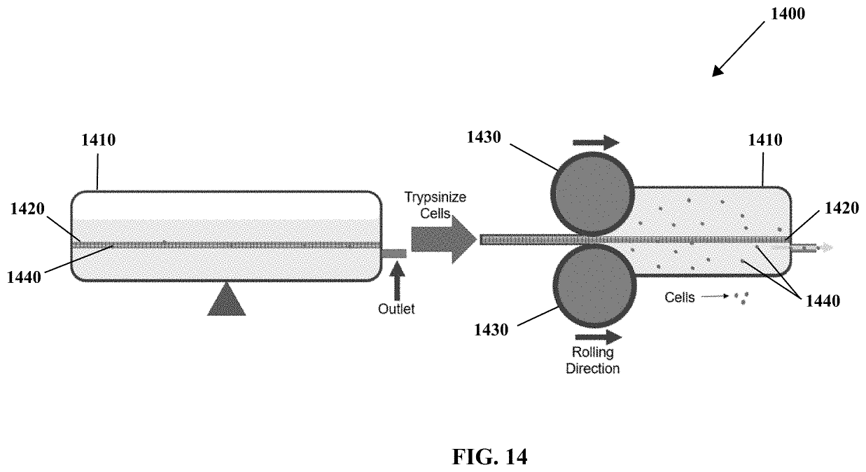

Turning to FIG. 14, a bioreactor system 1400 including a deformable bioreactor 1410 is used in conjunction with a textile cell growth matrix 1420 which can be mechanically deformed, such as with rollers 1430, to extract cells 1440 from the textile cell growth matrix 1420 following colonization. The cells on textile cell growth matrix 1420 may be trypsinized prior to contact with the rollers 1430 to assist with the release and collection of the cells 1440. In some embodiments, mechanical vibration, including by ultrasonic energy, may be directed to the textile cell growth matrix 1420 to further assist in the release of the cells 1440 from the textile cell growth matrix 1420. For example, the rollers 1430 may vibrate and or be configured to introduce ultrasonic energy during the rolling.

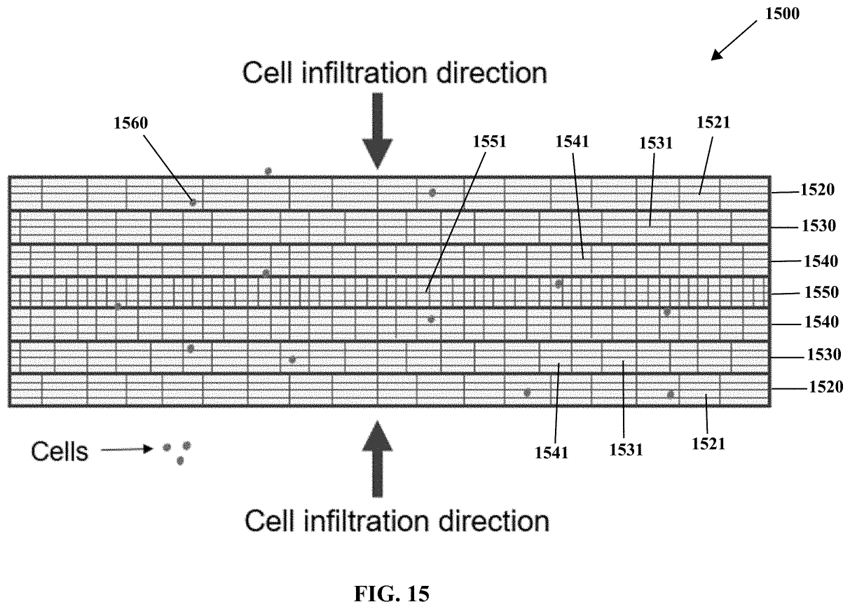

FIG. 15 illustrates a multilayer textile cell growth matrix 1500 with a gradient of porosity is shown. The multilayer textile cell growth matrix 1500 shown is of symmetrical construction and may be constructed by stacking individual growth matrices or as multiple layers of a continuous weave or knit.

In the embodiment of FIG. 15, an outer layer 1520 exhibits the largest average pore size of a first pore 1521. A second layer 1530 adjacent to the outer layer 1520 exhibits an average pore size of a second pore 1531 which is smaller than the average pore size of the first pore 1521. A third layer 1540 adjacent to the second layer 1530 exhibits an average pore size of a third pore 1541 which is smaller than the average pore size of the second pore 1531. A central layer 1550 adjacent to the third layer 1540 exhibits an average pore size of a fourth pore 1551 which is smaller than the average pore size of the third pore 1541. Cells 1560 may colonize or be trapped by some or all of the layers of the multilayer textile cell growth matrix 1500. In some embodiments, the outer textile layers of growth matrix include larger pore sizes to improve cellular infiltration into the core of the growth matrix.

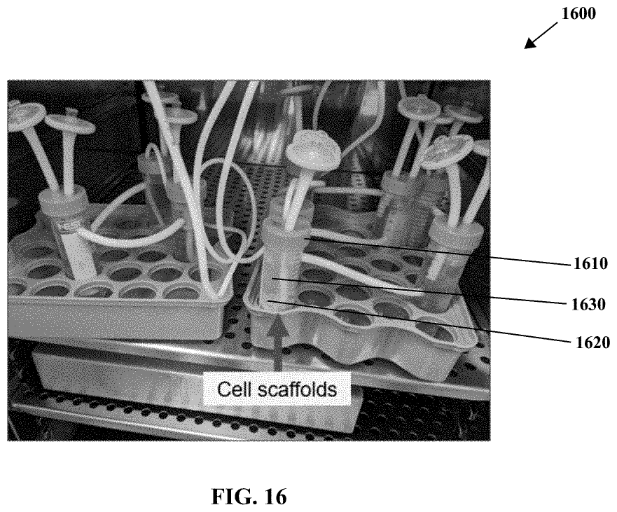

FIG. 16 shows a perfusion bioreactor system 1600 including bioreactors 1610 containing textile cell growth matrices 1620 in a nutrient rich medium 1630. In the example of FIG. 16, human cardiac fibroblasts (not shown) were cultured on textile disk growth matrices 1620. The cell cultures were maintained for six days under high media perfusion rates of 4.0 ml/min.

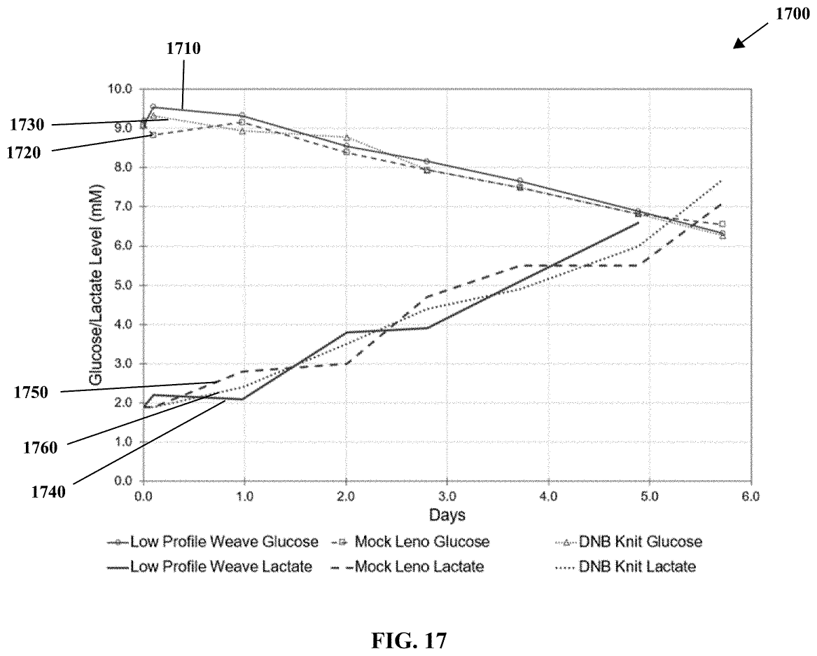

FIG. 17 is a graph 1700 which graphically represents the daily measurements of culture media glucose and lactate levels, which show cell proliferation on textile disk growth matrices as indicated by the data showing consumption of glucose and production of lactate by the cells. The data corresponds to the human cardiac fibroblast cultures presented in FIG. 16. In the examples represented by the graph 1700, curve 1710 represents the glucose concentration for cultures grown on a low-profile woven growth matrix, curve 1720 represents the glucose concentration for cultures grown on a mock leno weave growth matrix, and curve 1730 represents the glucose concentration for cultures grown on a double needle bar knit growth matrix. In the examples represented by the graph 1700, curve 1740 represents the lactate concentration for cultures grown on a low-profile woven growth matrix, curve 1750 represents the lactate concentration for cultures grown on a mock leno weave growth matrix, and curve 1760 represents the lactate concentration for cultures grown on a double needle bar knit growth matrix.

In some embodiments, mock leno structures may be preferred to provide control over porosity and overall solution properties. In some embodiments, growth matrices have a pore size in the range of 100 to 150 μm. This is sufficient to promote uniform cell culture media flow throughout all of the growth matrices used in a packed bed bioreactor. Additionally, the pore size is large enough to avoid being covered by cells as they expand and secrete their own extracellular matrix, ensuring that porosity remains for consistent solution flow for the lifetime of the growth matrix.

Growth matrix density may depend on size, but growth matrices of a 6 mm disk size may be used at a density of up to 30,000 growth matrices per liter per reactor. Upon sufficient levels of cellular expansion (or production of other byproducts such as virus or antibodies), cells would be removed from growth matrices prior to next steps of use. The growth matrix pore size is large enough at 100 to 150 μm to ease removal of cells once the culture process is complete by allowing trypsinized cells to more readily flow through the growth matrices. In some embodiments, growth matrices would be used for a single set of cell production and then be disposed of following removal of cells.

Large bioreactors, in which cells are located deeper within a packed bed structure, have limited access to the growth nutrients contained in media, textile disks with larger porosity can be used to promote better media flow throughout the entirety of the packed bed. This allows textile disks to be used in larger reactors than currently possible. Specific design and control of the orientation of the textiles used in the disks can promote improved growth of cells that respond to topographical cues for alignment, such as skeletal muscle cells or neural cells.

For high shear solution applications, which may be used to promote high levels of nutrient exposure, a layered weave can be used to provide ‘pockets’ into which cells can grow and be shielded from being detached from the substrate as a result of high solution shear forces. In some embodiments, the layered weave may result in variations in porosity within the growth matrix structure. In one embodiment, the porosity is less near the center of the growth matrix when compared to the surface and/or edges of the growth matrix. In one embodiment, a growth matrix having variations in porosity is formed as a mock leno weave construction.

A multi-layer growth matrix may also be used in high shear solution applications. The multi-layer growth matrix may include a plurality of knit or woven growth matrix layers. The multi-layer growth matrix may be constructed to result in variable porosity within the overall growth matrix. In one embodiment, the porosity is less near the center of the growth matrix when compared to the surface and/or edges of the growth matrix. In one embodiment, the portion of the growth matrix having reduced porosity includes a mock leno weave construction.

Growth matrices in accordance with embodiments described herein can be used for producing artificial skin, wound care applications, as a template for cartilage repair and regrowth, loaded with drug for use as a controlled release system, and used in biofiltration applications, among other applications. The growth matrices may also be used for cell-based therapy applications where a patient's own cells are expanded and later re-introduced back into the patient either separated from the growth matrices or with the growth matrices.

Growth matrices in accordance with embodiments described herein, may also be used to culture biological materials other than cellular materials. In some embodiments, the growth matrices may be used for culture of cells used to produce viruses. In some embodiments, the growth matrices may be used for the culture of cells used to produce therapeutic proteins or other biologics. In a further embodiment, the growth matrices may be used for bacteria and archaea production. It will further be appreciated that in some embodiments, growth matrices may be used for adsorbing cells to the material surface of the growth matrix for temporary permanence for exposure to vectors for gene-cell based therapy.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. An engineered textile construction comprising:
a first textile having a first average pore size forming a textile cell growth matrix;
wherein the first textile is a woven or a knit construction;
wherein the textile cell growth matrix is configured to have a surface area sufficient to promote cell expansion; and
wherein the first average pore size is preselected to prevent filling of the pores during cell expansion.

2. The textile cell growth matrix system of claim 1:
wherein fibers of the first textile are coated with a continuous or discontinuous resorbable material.

3. The engineered textile construction system of claim 2:
wherein the resorbable material includes at least one of polycaprolactone (PCL), polylactic acid (PLA), polyglycolic acid (PGA), poly(glycerol sebacate) (PGS), lysine-poly(glycerol sebacate) (KPGS), poly(glycerol sebacate urethane) (PGSU), amino-acid incorporated PGS, or acrylated poly(glycerol sebacate) (PGSA).

4. The engineered textile construction system of claim 2:
wherein the first textile layer comprises resorbable fibers.

5. The engineered textile construction system of claim 4:
wherein the resorbable fibers include at least one of polycaprolactone (PCL), polylactic acid (PLA), polyglycolic acid (PGA), poly(lactic-co-glycolic acid) (PLGA), poly(glycerol sebacate) (PGS), lysine-poly(glycerol sebacate) (KPGS), acrylated poly(glycerol sebacate) (PGSA), poly(trimethylene carbonate) (PTMC), poly(dioxanone) (PDO), collagen, fibrin, alginate, or silk.

6. The engineered textile construction of claim 1:
wherein the first textile consists of resorbable fibers.

7. The engineered textile construction system of claim 1:
wherein the textile cell growth matrix has a porosity of 5 percent to 75 percent.

8. The engineered textile construction system of claim 7:
wherein the textile cell growth matrix has a porosity of 40 percent to 75 percent.

9. The engineered textile construction system of claim 7:
wherein the textile cell growth matrix has a porosity of 5 percent to 40 percent.

10. The engineered textile construction system of claim 1:
wherein the engineered textile construction is pre-seeded with the at least one culturable cell type.

11. The engineered textile construction system of claim 1:
wherein the textile cell growth matrix exhibits a surface area of 0.01 m²/g to 10.0 m²/g.

12. The engineered textile construction system of claim 11:
wherein the textile cell growth matrix exhibits a surface area of 0.1 m²/g to 3.0 m²/g.

13. The engineered textile construction of claim 1 further comprising a second textile having a second average pore size, wherein the second textile is a woven or a knit construction and wherein the second average pore size is preselected to prevent filling of the pores during cell expansion.

14. The engineered textile construction of claim 13:
wherein the first average pore size of the first textile is different from the second average pore size of the second textile.

15. A cell culture system comprising:
a container comprising:
at least one culturable cell type;
a cell culture medium; and
an engineered textile construction according to claim 1.

16. The cell culture system of claim 15:
wherein the at least one culturable cell type includes a non-self-aggregating cell type.

17. The cell culture system of claim 16:
wherein the non-self-aggregating cell type is epithelial cells.

18. The cell culture system of claim 15:
wherein the at least one culturable cell type includes a self-aggregating cell type.

19. The cell culture system of claim 18:
wherein the self-aggregating cell type is selected from the list consisting of neural stem cells, mesenchymal stem cells, tumor cells, mesenchymal stem cells, pancreatic islet cells, and induced pluripotent stem cells, hepatocytes, and combinations thereof.

20. The cell culture system of claim 15:
wherein the engineered textile construction includes polyethylene terephthalate fibers coated with a resorbable material.

21. The cell culture system of claim 15:
wherein the engineered textile construction further includes a second textile having a second average porosity stacked with the first textile.

22. The cell culture system of claim 15:
wherein at least one textile layer of the engineered textile construction is pre-seeded with the at least one culturable cell type.

23. The cell culture system of claim 16:
wherein the engineered textile construction exhibits a surface area of 0.01 m²/g to 10.0 m²/g.

24. The cell culture system of claim 23:
wherein the engineered textile construction exhibits a surface area of 0.1 m²/g to 3.0 m²/g.

25. An implantable device comprising:
a knit or woven textile having a first average pore size;
wherein the first average pore size is preselected to prevent filling of the pores during colonization;
wherein the porosity is about 40 percent to about 70 percent;
wherein the textile includes a resorbable material; and
wherein the textile is pre-seeded with at least one culturable cell type.
